(19) United States
(12) Patent Application Publication
(10) Pub. No.: US 2026/0130577 A1
(43) Pub. Date: May 14, 2026

(54) DISTANCE RETAINER FOR USE IN ENDOSCOPIC PROBE TUBES

(71) Applicant: MEDICAL INTUBATION TECHNOLOGY CORPORATION, Taoyuan City (TW)

(72) Inventor: Hsin Yun Tsai, Tainan City (TW)

(21) Appl. No.: 19/360,667

(22) Filed: Oct. 16, 2025

(30) Foreign Application Priority Data
Oct. 17, 2024 (TW) 113139569

(51) Int. Cl. A61B 1/00

(52) U.S. Cl. CPC A61B 1/00147

(57) ABSTRACT

A spacer for an endoscope probe tube includes a central sleeve configured for an endoscope probe tube to pass through and to be sleeved onto and fixed to the endoscope probe tube, the central sleeve having a circumferential surface; and an outer member rotatably sleeved on the central sleeve such that the outer member is freely rotatable relative to the circumferential surface of the central sleeve under an external force. The outer member has at least one expansion portion extending radially outward from the endoscope probe tube by a predetermined distance. The at least one expansion portion is configured to abut against an external ground surface or a surface of an object to be observed, so as to maintain a predetermined distance between the endoscope probe tube and the ground surface or the surface of the object to be observed.

### FIELD OF THE INVENTION

The present disclosure relates to endoscopes, and more particularly to a spacer configured for use with an endoscope probe tube to enable the endoscope probe tube to maintain a predetermined distance from a ground surface or the surface of an object to be observed.

### PRIOR ART

Taiwan Patent No. I590878 discloses an endoscope guiding accessory that enables the head end of an endoscope tube to stay or move along a pipeline steadily, allows its first guide member to be sleeved on the endoscope body and its second guide member through which the endoscope tube passes, and uses its flexible member to connect the first guide member and the second guide member. In practice, the first guide member is surrounded by a radially extending first support portion, which keeps the head end of the endoscope tube at a predetermined distance from the surface of the object being observed, thereby preventing degradation of the field of view caused by the lens being too close to a wall surface. Furthermore, the first guide member compresses the flexible member to cause the deformation thereof while the first guide member is passing through a bend inside the pipe, so as to generate a pushing force for pushing the first guide member to cause its departure from the bend and thereby prevent the endoscope guiding accessory from getting stuck in the bend.

In the course of using an endoscope to perform observation, the endoscope tube must bend or rotate within narrow gaps or pipelines in order to perform observation, and such bending can also cause rotation of the endoscope tube. When the endoscope guiding accessory is installed, since the first guide member of the prior art is detachably sleeved on the endoscope tube and, when the first guide member is sleeved on the endoscope tube, is fixed relative thereto, rotation of the endoscope tube during observation requires concomitant rotation of the endoscope guiding accessory.

However, during actual observation, a portion of the endoscope guiding accessory contacts or abuts against an inner wall or bend of the pipe, making it difficult to rotate. Moreover, since the endoscope guiding accessory is usually larger and heavier than the endoscope tube itself, the user has to overcome the resistance arising from the contact and friction between the endoscope guiding accessory and the pipe wall or bend in order to rotate the endoscope tube. As a result, it is difficult or even impossible for the user to rotate the endoscope tube.

The present invention aims to address the foregoing rotation issue facing endoscope tubes in the presence of the endoscope guiding accessory.

### SUMMARY OF THE INVENTION

It is an objective of the disclosure to provide a spacer configured for use with an endoscope probe tube to allow the endoscope probe tube, during use, to not only maintain a predetermined distance from the surface of an object being observed but also to be easily rotated.

To achieve the above and other objectives, the disclosure provides a spacer for an endoscope probe tube, comprising: a central sleeve configured for an endoscope probe tube to pass through and to be sleeved onto and fixed to the endoscope probe tube, the central sleeve having a circumferential surface; and an outer member rotatably sleeved on the central sleeve such that the outer member is freely rotatable relative to the circumferential surface of the central sleeve under an external force, the outer member having at least one expansion portion extending radially outward from the endoscope probe tube by a predetermined distance. The at least one expansion portion is configured to abut against an external ground surface or a surface of an object to be observed, so as to maintain a predetermined distance between the endoscope probe tube and the ground surface or the surface of the object to be observed.

Therefore, the disclosure provides a spacer configured for use with an endoscope probe tube to allow the endoscope probe tube, during use, to not only maintain a predetermined distance from the surface of an object being observed but also to be easily rotated.

### BRIEF DESCRIPTION OF THE DRAWINGS

FIG. 1 is a perspective view of a spacer for an endoscope probe tube according to the first preferred embodiment of the disclosure.

FIG. 2 is an exploded view of the spacer for an endoscope probe tube according to the first preferred embodiment of the disclosure.

FIG. 3 is a front view of the spacer for an endoscope probe tube according to the first preferred embodiment of the disclosure.

FIG. 4 is a cross-sectional view of the spacer for an endoscope probe tube taken along line 4-4 of FIG. 3.

FIG. 5 is an assembled schematic view of the spacer for an endoscope probe tube according to the first preferred embodiment of the disclosure.

FIG. 6 illustrates the state of use of the spacer for an endoscope probe tube according to the first preferred embodiment of the disclosure.

FIG. 7 illustrates another usage state of the spacer for an endoscope probe tube according to the first preferred embodiment of the disclosure.

FIG. 8 is a perspective view of another spacer for an endoscope probe tube according to the second preferred embodiment of the disclosure.

FIG. 9 illustrates another state of use of the second preferred embodiment of the disclosure.

FIG. 10 illustrates yet another embodiment of the disclosure.

FIG. 11 illustrates still another embodiment of the disclosure.

FIG. 12 illustrates a further embodiment of the disclosure.

### IMPLEMENTATION

Objectives, technical features, and advantages of the disclosure are herein illustrated with preferred embodiments, depicted with drawings, and described below.

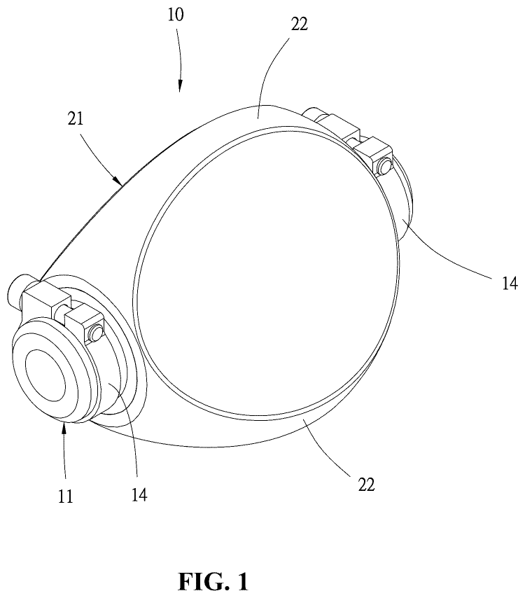

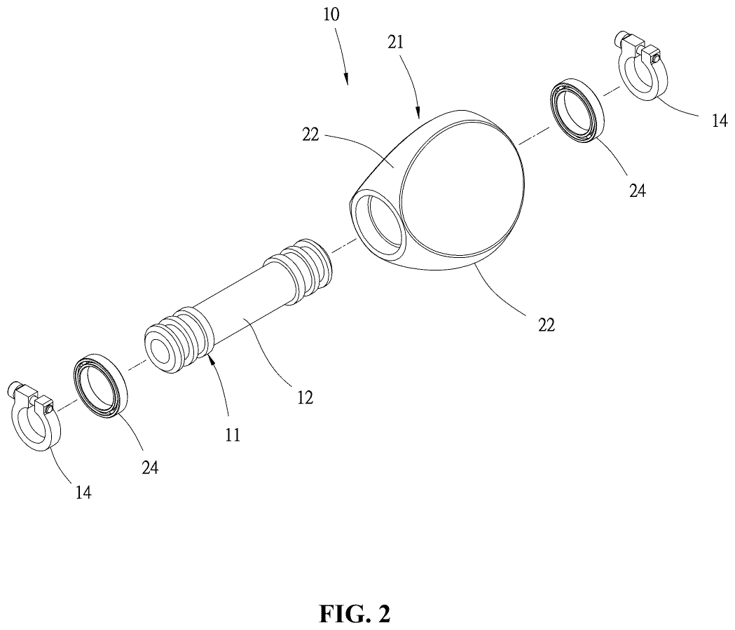

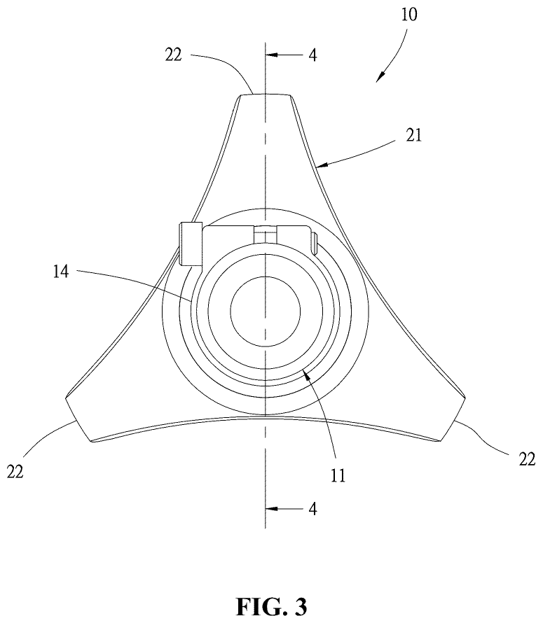

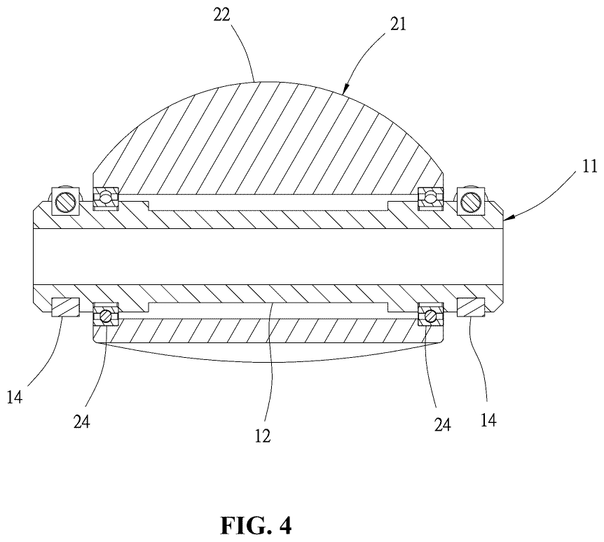

Referring to FIG. 1 through FIG. 4, the first preferred embodiment of the disclosure provides a spacer 10 for an endoscope probe tube. The spacer 10 essentially comprises a central sleeve 11 and an outer member 21.

The central sleeve 11 is configured for an endoscope probe tube 91 to pass through and to be sleeved onto and fixed to the endoscope probe tube 91. The central sleeve 11 has a circumferential surface 12. In the first embodiment, the central sleeve 11 is made of an elastic material such as rubber and is clamped and fixed onto the endoscope probe tube 91 by two clamping rings 14 disposed around the circumferential surface 12 of the central sleeve 11. The endoscope probe tube 91 comprises a head end and a flexible tube that are well-known among those skilled in the art but is depicted as one single tube in the accompanying drawings.

The outer member 21 is rotatably sleeved on the central sleeve 11 and can freely rotate relative to the circumferential surface 12 of the central sleeve 11 under external force. The outer member 21 has three expansion portions 22 extending radially outward from the endoscope probe tube 91 by a predetermined distance, the three expansion portions 22 being evenly spaced at equal angular intervals around the outer member 21. In the first embodiment, the outer member 21 is rotatably mounted on the central sleeve 11 by two bearings 24, thereby allowing the outer member 21 to freely rotate relative to the central sleeve 11.

The three expansion portions 22 are configured to abut against an external ground surface or the surface of an object to be observed so as to maintain a predetermined distance between the endoscope probe tube 91 and the ground surface or the surface of an object to be observed. In the first embodiment, the external ground surface or the surface of an object to be observed is exemplified by an inner wall 99 of a pipe.

The structural features of the first embodiment are described above. The state of use of the first embodiment is described below.

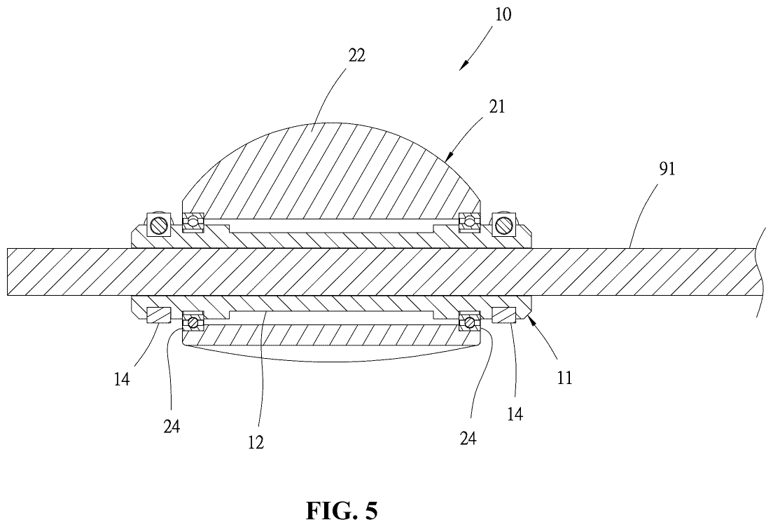

As shown in FIG. 5, before use, the spacer 10 of the first embodiment is mounted on the endoscope probe tube 91 by inserting the endoscope probe tube 91 through the central sleeve 11 and tightening the two clamping rings 14 to fix the central sleeve 11 to the endoscope probe tube 91. Since the outer member 21 is already supported on the central sleeve 11 through the two bearings 24, the outer member 21 is positioned between the two clamping rings 14 and remains freely rotatable relative to the central sleeve 11.

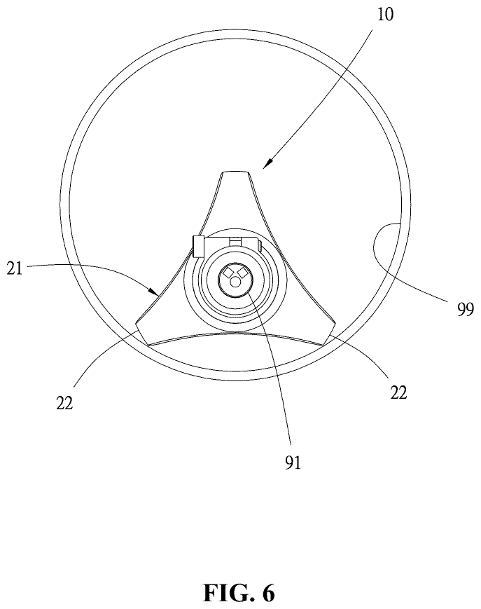

As shown in FIG. 6, during use, the user inserts the endoscope probe tube 91 and the spacer 10 into a pipe to be observed. At this time, the spacer 10 rests against the inner wall 99 of the pipe through two of its three expansion portions 22 under the action of gravity. The endoscope probe tube 91 is at a predetermined distance from the inner wall 99 because of the structural features of the two expansion portions 22. With the outer member 21 being freely rotatable relative to the central sleeve 11, the user can readily rotate the endoscope probe tube 91, which in turn rotates the central sleeve 11 without causing rotation of the outer member 21, thereby facilitating smooth rotation of the endoscope probe tube 91. For an endoscope equipped with a side-view lens, a full 360-degree rotational observation can be easily achieved, making it particularly suitable for inspecting welding conditions or cracks inside a pipe. Accordingly, the first embodiment effectively overcomes the drawbacks of the prior art.

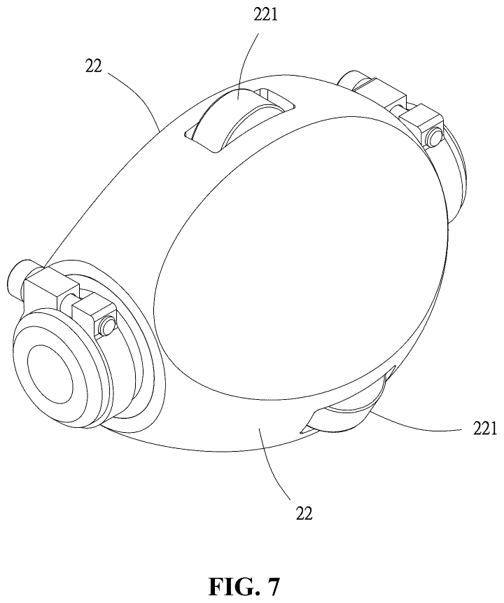

As shown in FIG. 7, in the first embodiment, each of the expansion portions 22 is provided, at a position radially outward of the central sleeve 11, with a roller 221 configured to abut against the inner wall 99 of the pipe (as shown in FIG. 6). Each roller 221 can roll during movement to reduce friction, thereby providing smoother movement and reducing operational resistance during manipulation. Each of the expansion portions 22 is provided, at a position radially outward of the central sleeve 11, with a roller 221 configured to abut against the inner wall 99 (shown in FIG. 6). Each roller 221 can roll during movement to reduce friction, thereby enabling smoother movement and reducing operational resistance during movement manipulation.

Therefore, the first embodiment, during use, the endoscope probe tube 91 is maintained at a predetermined distance from the surface of the object to be observed (i.e., the inner wall 99), thereby facilitating imaging and observation; moreover, the endoscope probe tube 91 can be readily rotated without experiencing any difficulty in rotation.

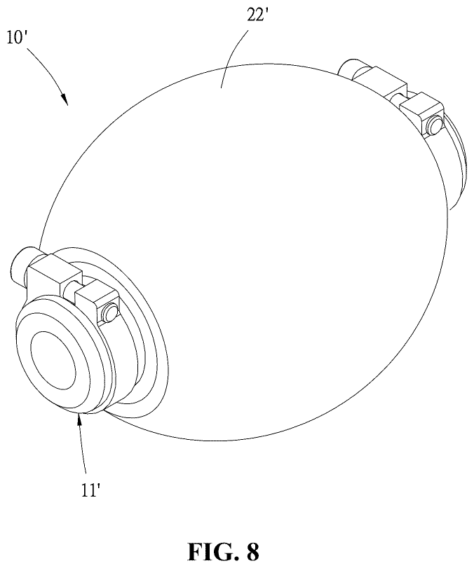

As shown in FIG. 8, the second preferred embodiment of the disclosure provides a spacer 10′ for an endoscope probe tube, which is substantially similar to the foregoing first embodiment, except that:
Unlike the first embodiment which provides three expansion portions 22, the second embodiment provides one expansion portion 22′.

In the second embodiment, the expansion portion 22′ is circular in a radial cross section of the central sleeve 11′. In addition, the expansion portion 22′ has a smaller diameter at both ends of the central sleeve 11′ and a larger diameter at the middle portion thereof, thereby forming an ellipsoidal shape.

During use, the second embodiment directly brings a portion of the expansion portion 22′ into abutment with an external ground surface or the surface of an object to be observed, namely the inner wall 99 (shown in FIG. 6), while still maintaining a state in which the expansion portion 22′ is spaced a predetermined distance from the inner wall 99.

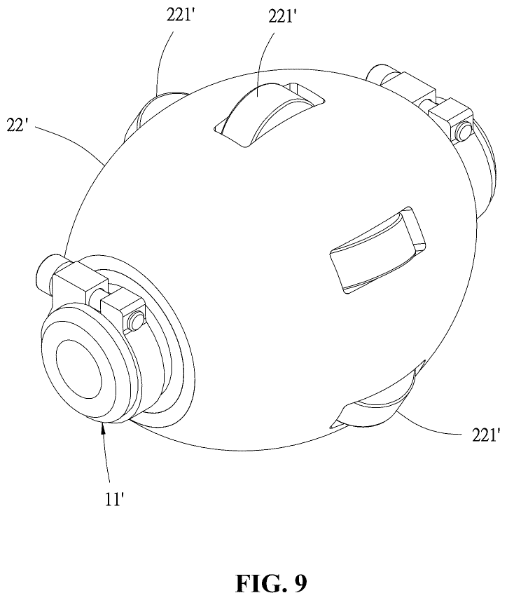

As shown in FIG. 9, the expansion portion 22′ may also be provided with a plurality of rollers 221′ so as to reduce movement resistance encountered during operation.

The remaining structure and the effects achieved by the second embodiment are substantially similar to those of the foregoing first embodiment and will therefore not be described further.

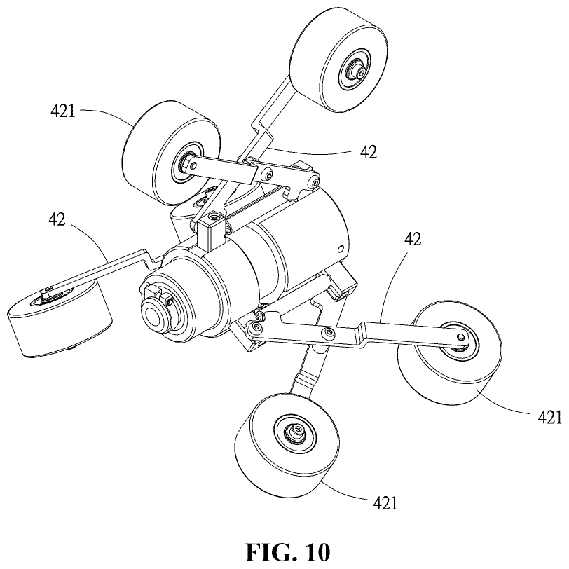

As shown in FIG. 10, each expansion portion 42 of the disclosure may alternatively be formed as an outwardly extending bracket provided with a plurality of rollers 421 configured to abut against the inner wall (not shown).

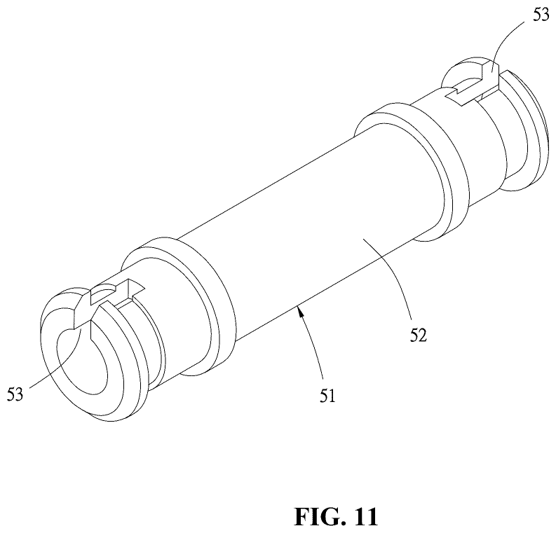

As shown in FIG. 11, the central sleeve 51 of the disclosure may also be made of metal and provided with axial slots 53 at both ends. The axial slots 53 extend axially. Two clamping rings (not shown) disposed around the circumferential surface 52 of the central sleeve 51 and corresponding in position to the axial slots 53 are tightened, such that the central sleeve 51 can be securely fixed onto the endoscope probe tube (not shown).

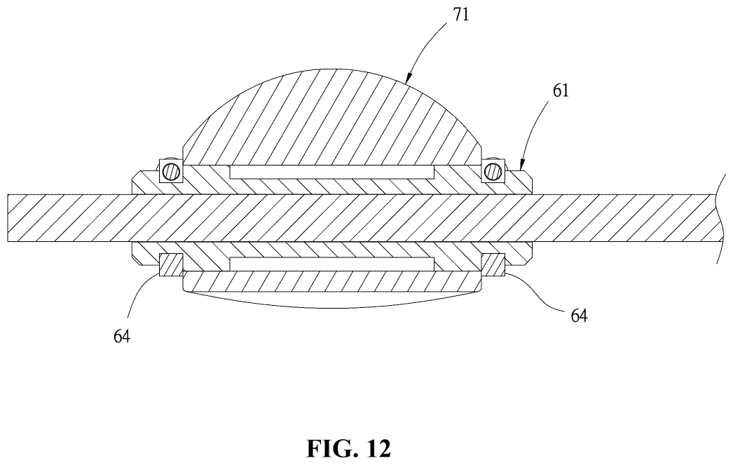

As shown in FIG. 12, a variant embodiment of the disclosure dispenses with the two bearings 24 of the first embodiment of the disclosure and provides the two clamping rings 64 configured to restrain the outer member 71 such that it remains rotatably sleeved around the central sleeve 61 without detachment. At this time, the outer member 71 is loosely fitted on the central sleeve 61 and can rotate freely relative to the central sleeve 61. Although the rotational smoothness of the outer member 71 relative to the central sleeve 61 is inferior to that achieved by the bearings 24 of the first embodiment, it still meets user requirements and achieves the intended free rotation. Therefore, the foregoing technical features fall within the scope of the claims of the disclosure.

The disclosure is disclosed above by embodiments. However, persons skilled in the art should understand that the embodiments are illustrative of the disclosure only, but shall not be interpreted as restrictive of the scope of the disclosure. Please note that all simple variations and equivalent implementation of the embodiments shall be deemed to fall within the scope of the claims of the disclosure.

### Explanation of Symbols

10: spacer
11: central sleeve
12: circumferential surface
14: clamping ring
21: outer member
22: expansion portion
221: roller
24: bearing
42: expansion portion
421: roller
51: central sleeve
52: circumferential surface
53: axial slot
61: central sleeve
64: clamping ring
71: outer member
91: endoscope probe tube
99: inner wall
10′: spacer
11′: central sleeve
22′: expansion portion
221′: roller

1. A spacer for an endoscope probe tube, comprising:
a central sleeve configured for an endoscope probe tube to pass through and to be sleeved onto and fixed to the endoscope probe tube, the central sleeve having a circumferential surface; and
an outer member rotatably sleeved on the central sleeve such that the outer member is freely rotatable relative to the circumferential surface of the central sleeve under an external force, the outer member having at least one expansion portion extending radially outward from the endoscope probe tube by a predetermined distance,
wherein the at least one expansion portion is configured to abut against an external ground surface or a surface of an object to be observed, so as to maintain a predetermined distance between the endoscope probe tube and the ground surface or the surface of the object to be observed.

2. The spacer for an endoscope probe tube according to claim 1, wherein the central sleeve is made of an elastic material and is clamped and fixed onto the endoscope probe tube by two clamping rings disposed around the circumferential surface of the central sleeve.

3. The spacer for an endoscope probe tube according to claim 2, wherein the two clamping rings are configured to restrain the outer member such that the outer member remains rotatably sleeved around the central sleeve without detachment.

4. The spacer for an endoscope probe tube according to claim 1, wherein the central sleeve is made of a metallic material and has axial slots at both ends thereof, the central sleeve being clamped and fixed onto the endoscope probe tube by two clamping rings disposed around the circumferential surface of the central sleeve at positions corresponding to the two axial slots.

5. The spacer for an endoscope probe tube according to claim 1, wherein the outer member is rotatably mounted on the central sleeve by two bearings.

6. The spacer for an endoscope probe tube according to claim 5, wherein the at least one expansion portion comprises a plurality of expansion portions evenly spaced at equal angular intervals around the outer member.

7. The spacer for an endoscope probe tube according to claim 6, wherein each of the expansion portions is provided, at a position radially outward of the central sleeve, with a roller configured to abut against an external ground surface or a surface of an object to be observed.

8. The spacer for an endoscope probe tube according to claim 6, wherein each of the expansion portions is an outwardly extending bracket provided with a plurality of rollers configured to abut against an external ground surface or a surface of an object to be observed.

9. The spacer for an endoscope probe tube according to claim 5, wherein the at least one expansion portion comprises one expansion portion being circular in a radial cross section of the central sleeve.

10. The spacer for an endoscope probe tube according to claim 9, wherein the expansion portion has a smaller diameter at both ends of the central sleeve and a larger diameter at a middle portion thereof, thereby forming an ellipsoidal shape.
